(19) United States
(12) Patent Application Publication
(10) Pub. No.: US 2026/0284356 A1
(43) Pub. Date: Sep. 24, 2026

(54) FUNCTIONAL MAINTENANCE OF CARDIOVASCULAR CATHETERS

(71) Applicant: The Cleveland Clinic Foundation, Cleveland, OH (US)

(72) Inventors: Jamshid KARIMOV, Cleveland Heights, OH (US); Kiyotaka FUKAMACHI, Mayfield Heights, OH (US)

(21) Appl. No.: 19/476,324

(22) PCT Filed: Apr. 19, 2024
(86) PCT No.: PCT/US2024/025358

§ 371 Date: Oct. 17, 2025

Related U.S. Application Data
(60) Provisional application No. 63/460,763, filed on Apr. 20, 2023.

(51) Int. Cl. A61M 25/00; A61M 39/02

(52) U.S. Cl. CPC A61M 25/0097; A61M 39/0247; A61M 2025/0019; A61M 2039/0258; A61M 2205/103

(57) ABSTRACT

A system for minimizing nucleation of clot and biofilm material on inner and outer surfaces of a medical tube a hub connector and a mechanical agitator. The hub connector is configured to provide fluid communication between a medical tube and one or more fluid sources when attached thereto. The mechanical agitator is associated with the hub connector and is configured to deliver oscillatory mechanical energy thereto so that the oscillatory mechanical energy further will be delivered substantially uniformly to the medical tube when attached thereto.

### CROSS-REFERENCE TO RELATED APPLICATION

This application claims the benefit of US Provisional Patent Application Ser. No. 63/460,763 filed Apr. 20, 2023, whose contents are incorporated by reference.

### TECHNICAL FIELD

The present disclosure relates generally to systems and methods for minimizing nucleation of clot and biofilm material on inner and outer surfaces of a medical tube, specifically cardiovascular catheters.

### BACKGROUND

Cardiovascular catheters are routinely used in patients to provide access to large central veins and arteries near the patient's heart during emergencies or for long-term medical treatment. Such catheters allow medication, fluids or blood to be supplied directly and quickly to the patient's circulatory system, as well as the withdrawal of blood for testing. Such catheters also avoid the pain or discomfort associated with frequent needle sticks, and reduce the risk of irritation to smaller peripheral vasculature in the patient's extremities or other parts of the body. These catheters also allow measurement of hemodynamic variables that cannot be accurately quantified by noninvasive means. A peripherally inserted central venous catheter (PICC) is one example of such a catheter. A PICC line is usually inserted into a vein in the patient's neck, chest, arm or groin and is passed through the vein until the tip of the PICC line is positioned in the vena cava. FIG. 1 shows a PICC line inserted via a brachial vein in the patient's upper arm. A central venous catheter (CVC) is similar to a PICC line except that it is usually inserted via, e.g. the subclavian vein (seen in FIG. 5).

Catheters, including CVC and PICC lines, may become clogged by substances that pass through the line, such as clotted blood. In standard practice, catheter patency maintenance consists of periodic and frequent flushing of the catheter with a solution. Additionally, biofilm and/or blood clots may form along the outer surface of the catheter and at the tip, which can lead to life threatening complications such as increased risks of infection, vein or arterial thrombosis or pulmonary embolism. These complications can compromise the ability to provide medication, fluids or blood and to draw blood and measure hemodynamic parameters, as well as adversely influence surgical outcomes or delay treatment and/or recovery.

Despite many improvements in catheter maintenance and care, clotting in and around catheters, as well as biofilm formation, remain important contributors to adverse effects on patients' treatment. In most cases, clotting within the catheter lumen is prevented or treated by KVO (“keep vein open”) infusions, which requires putting a low volume of infusion fluid through the catheter continuously to diminish aggregation of clotting material in the lumen, (e.g. tPA infusions, saline) to break up a clot already formed, or periodic flushing of the catheter. If these measures fail, the patient must undergo a specialized vascular or endovascular procedure, or surgery to remove clots that may have formed in or around the catheter, in the vein in which the catheter is placed and/or in or around the heart, and to replace the catheter. There is currently no reliable method to prevent clots from forming and/or attaching inside the catheter or on its outer surface, or the formation of biofilm buildup inside or on the outer surface.

### SUMMARY

Disclosed herein are systems and methods that can be utilized to minimize formation nucleation of clot and/or biofilm material on inner and outer surfaces of a medical tubing, such as a catheter. As will be further described, the system can utilize a mechanical agitator integrated with a hub connector attached to the proximal end of the tube outside of the patient. The position of the mechanical agitator on the hub connector enables the conduction of mechanical motion to the hub connector and from there along the entire length of the medical tube extending therefrom toward and into the patient. The delivery of this mechanical motion along the entire length of the medical tube minimizes or prevents the initial formation or accumulation of clot along the entire outer surface length of the medical tube. Thus, the likelihood of clot formation and growth is reduced. Further, the delivery of mechanical motion along the full length of the catheter, up to and including its tip in-use, minimizes or prevents the formation of obstructions in a lumen of the catheter to maintain its patency.

In an aspect, the present disclosure includes a system for minimizing formation of clot and/or biofilm material on inner and outer surfaces of a medical tube, a hub connector and a mechanical agitator. The hub connector is configured to provide fluid communication between a medical tube and one or more fluid sources when attached thereto. The mechanical agitator is integrated with the hub connector and is configured to deliver oscillatory mechanical energy thereto so that the oscillatory mechanical energy further will be delivered along the entire length of the medical tube when attached thereto.

In another aspect, a method of minimizing formation of clot and/or biofilm material on inner and outer surfaces of a medical tube is provided. The method includes attaching a proximal end of the medical tube to a hub connector having a mechanical agitator integrated with the hub connector and configured to deliver oscillatory mechanical energy. The method also includes inserting a distal end of the medical tube into a blood vessel of a patient and activating the mechanical agitator to deliver oscillatory mechanical along entire length of the medical tube, via the hub connector, up to and including a tip thereof resident within the blood vessel.

### BRIEF DESCRIPTION OF DRAWINGS

The foregoing and other features of the present disclosure will become apparent to those skilled in the art to which the present disclosure relates upon reading the description with reference to the accompanying drawings, in which:

FIG. 1 illustrates a PICC line inserted via a brachial vein in a patient's upper arm.

FIG. 2 illustrates a top view of an embodiment of a catheter having a hub connector equipped with a mechanical agitator.

FIG. 2a illustrates a close-up view of the distal end of the catheter shown in FIG. 2.

FIG. 3 illustrates a cross-sectional view of a catheter having two lumen.

FIG. 4 illustrates a side perspective view of the catheter of FIG. 2, close up on the hub connector equipped with the mechanical agitator.

FIG. 5 illustrates an embodiment of a system wherein a CVC catheter inserted the brachial vessel of a patient and the mechanical agitator is operative.

FIG. 6A illustrates clotting observed along the outer surface of the tip and the distal end portion of a control group catheter tested in Example 1.

FIG. 6B illustrates clotting observed in a cut-away segment of the lumen in the distal end portion of a control group catheter tested in Example 1.

FIG. 6C illustrates clotting observed in cross-sectional segments of the lumen of a control group catheter tested in Example 1.

FIG. 7A illustrates a lack of clotting observed in a cut-away segment of the lumen of an experimental group catheter tested in Example 1.

FIG. 7B illustrates a lack of clotting observed in a cut-away segment of lumen in the proximal end of an experimental group catheter tested in Example 1.

FIG. 7C illustrates a lack of clotting observed in cross-sections of segments of the lumen of an experimental group catheter tested in Example 1.

FIG. 8 compares average acceleration data observed at the tip and the proximal end, respectively, of catheters tested according to Example 1.

FIGS. 9-11 depict, respectively, the specific orthogonal-component accelerations observed for the average accelerations graphed in FIG. 8, as measured in Example 1.

FIGS. 12 and 13 reflect additional measurements of average velocity and average displacement, respectively, obtained at the tip and the proximal end, respectively, of the catheters tested in Example 1.

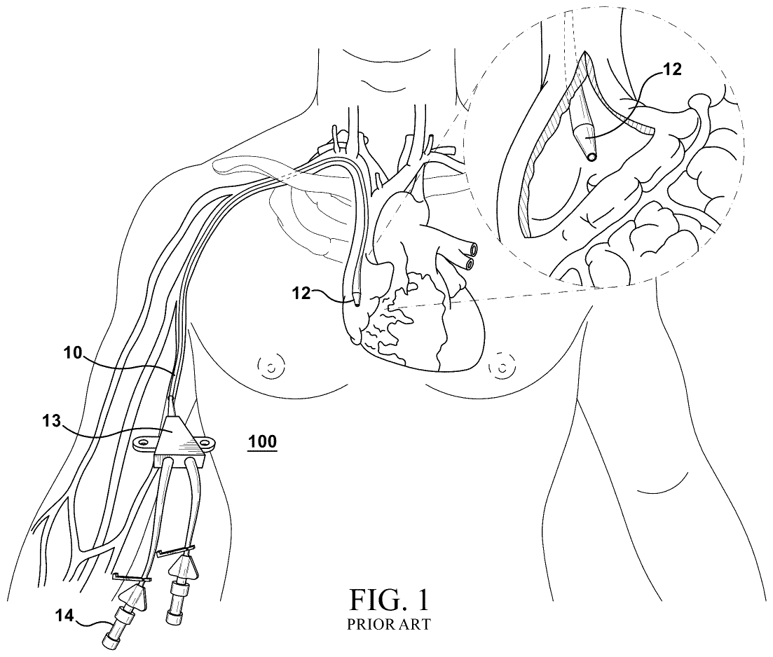

### DESCRIPTION

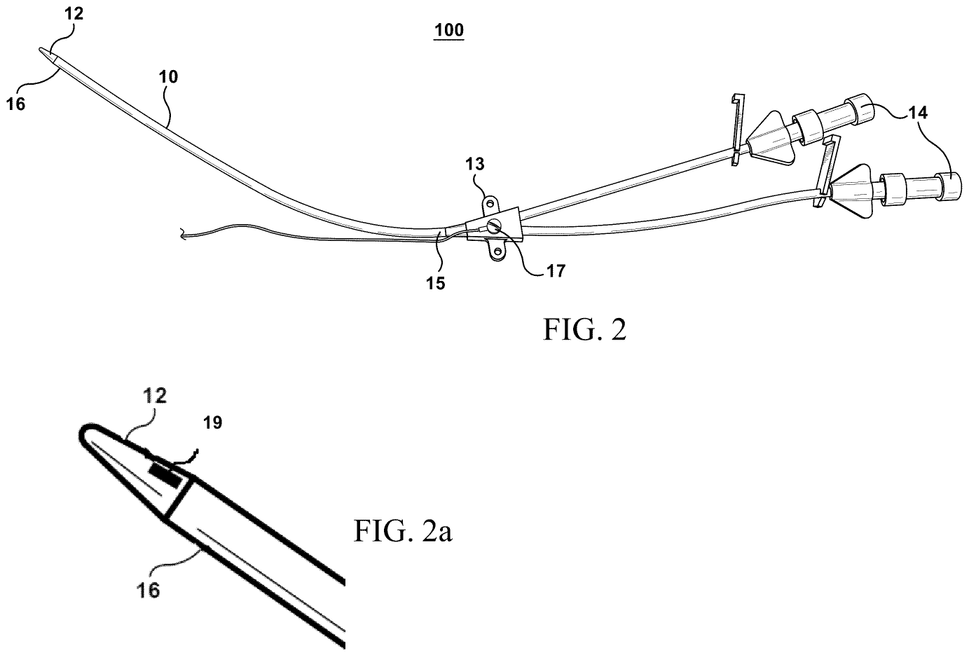

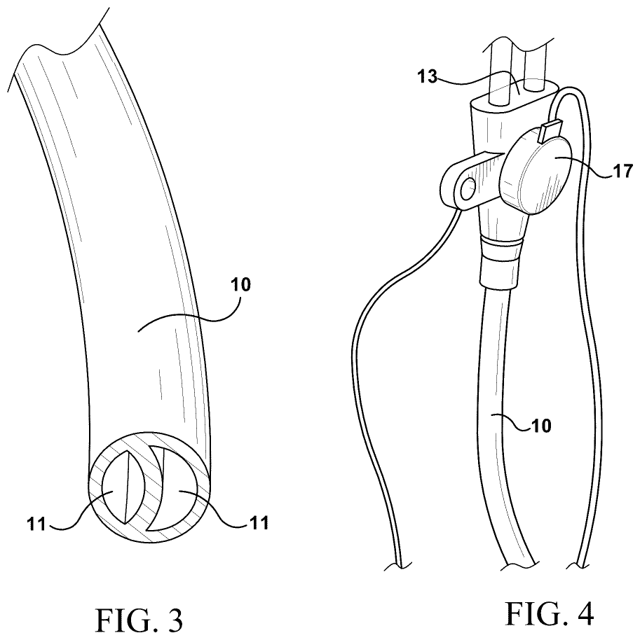

An aspect of the present disclosure can include a system for reducing the formation of blood clots and biofilm on the surface of a tube, such as a catheter. Referring first to FIG. 2, an embodiment of such a system 100 is shown. The system 100 includes a catheter 10 typically in the form of a tube having an inner lumen (not shown). The catheter 10 can be a CVC or a PICC line, for example. The catheter 10 is configured to be inserted into a patient's vein. The catheter 10 may include one or more lumens 11. For example, FIG. 3 illustrates a catheter 10 having two lumens 11. When the catheter 10 contains more than one lumens 11, each lumen 11 may be configured to transport a different fluid (e.g., medicine, nutrient or blood).

As explained in further detail below, the transmission of vibration along the catheter 10 from a mechanical agitator 17 may be affected by the dimensions of the catheter 10, including its length and diameter. The dimensions of the catheter 10 may vary depending on the specific clinical scenario or based on user preference. For example, the catheter 10 can have the diameter of a standard CVC line, for example 3-10 Fr. In certain embodiments, the catheter 10 has a diameter of about 5 Fr to about 8 Fr. In some embodiments, the diameter of the catheter 10 may be uniform throughout the length of the catheter. In other embodiments, the diameter of the catheter 10 may vary along its length.

Further, the density of the catheter material may affect the transmission of vibration along the catheter 10. A more-dense material has more mass per volume than a less dense material. Mass and density govern the speed of vibration transmission and how transmitted vibrations lose energy over distance and time. Thus, the amplitude of vibration would differ in softer and stiffer portions of the catheter when the same vibrational force is applied. In one embodiment, the catheter 10 can be fabricated from a single material, for example, a soft polymer, such as flexible polyvinyl chloride, silicone or polyurethane, or a stiffer polymer, such as stiff polyvinyl chloride, polyethylene, polypropylene, polymethyl methacrylate, acrylonitrile butadiene styrene (ABS) or polycarbonate. In another embodiment, the catheter 10 can be fabricated from a non-polymer material, or from two or more materials. For example, the distal end 16 of the catheter 10 may be made of one material, while the proximal end 17 of the catheter 10 may be made of a second material. A third material may also be used in a middle portion of the catheter 10 between the proximal and distal ends. In another example, when the catheter 10 contains more than one lumen 11, each lumen 11 may be formed by walls or wall segments made of materials that are the same or different.

Returning to FIG. 2, a tip 12 is configured to be inserted through the patient's vein and can be positioned in the patient's vena cava or advanced further into the right atrium. The tip 12 contains an opening that provides a passage for delivering or removing one or more fluid(s) when placed. The tip may also be fabricated from the same soft polymer or other suitable material as the catheter 10, or it may be fabricated from a stiffer material than the catheter 10 that permits the tip 12 to more easily navigate through the patient's vein to the vena cava, for example a medical-grade plastic.

A hub connector 13 is configured to join one or more fluid source connectors 14 with the catheter 10. The hub connector 13 may similarly be fabricated from the same soft polymer as the catheter 10. Alternatively, the hub connector 13 may be fabricated from a stiffer material to secure the connection with the catheter 10 and the fluid source connector(s) 14, and impart stability to the catheter 100, such as a medical-grade plastic. In some embodiments, hub connector 13 is integrally attached to the catheter 10 and the fluid source connector(s) 14, providing an integrated system 100 comprising a hub connector 13, a catheter 10, and one or more fluid source connectors 14. In other embodiments, the hub connector 13 is configured to removably attach to the catheter 10 and/or to the fluid source connector(s) 14; the latter via fluid source lines as seen in FIG. 2. When the hub connector 13 is operatively connected to the catheter 10, a proximal end 17 of the catheter 10 communicates with the hub connector 13 and a distal end 16 of the catheter 10 terminates in the tip 12. When connected, fluid from the fluid source connector(s) 14 passes through their respective tubing or lines into one or more respective lumens 11 in the catheter 10 via the hub connector 13. Accordingly, one or more fluids can be passed from separate fluid source connectors 14 into one catheter 10 for delivery to the vena cava without comingling the fluids.

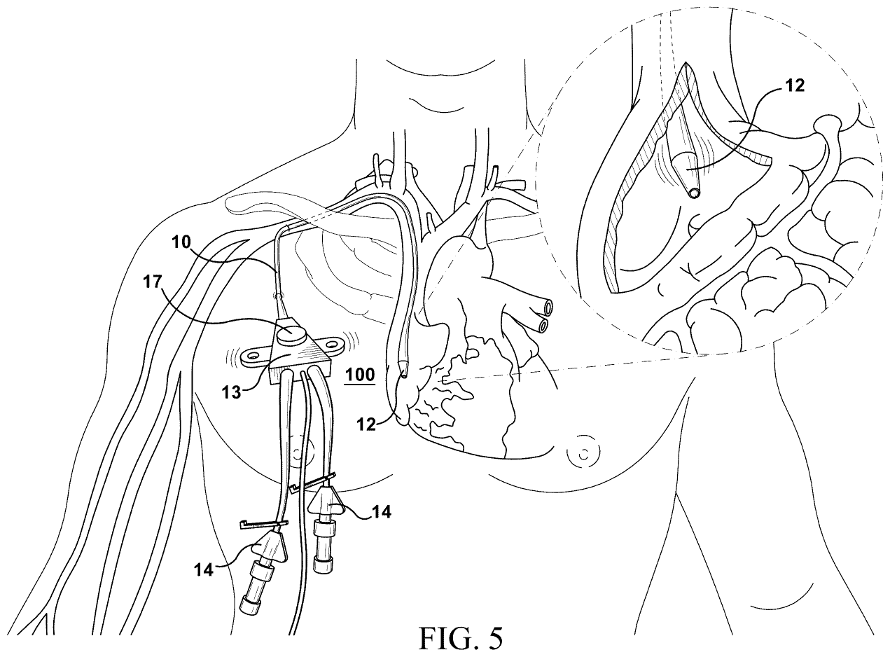

The hub connector 13 is equipped with a mechanical agitator 17. In the embodiments shown in FIGS. 2 and 4, the mechanical agitator 17 is a separately manufactured device fixed to the outer surface of the hub connector 13. In the embodiment shown in FIG. 5, the mechanical agitator 17 is integrated into the hub connector 13. The mechanical agitator 17 is effective to introduce (preferably oscillatory) mechanical motion (e.g. vibration or oscillation) to and along the length fo the catheter as will be further described, and is electrically coupled to an electrical energy source (not shown) in an operative state. Preferably, the electrical energy source is self-contained. In one example, the mechanical agitator 17 may include a motor having an eccentric mass affixed or connected to its crank shaft. The motor may be, for example, a coin motor or an ERM (cylindrical) motor. As illustrated in FIGS. 2 and 4, the mechanical agitator 17 is a coin motor. In some embodiments, the mechanical agitator 17 can include a motor with ball bearings that can be sealed, and a controller. In other embodiments, the motor can have various speeds such as speeds of about 1 rpm to about 25,000 rpm, about 10 rpm to about 2,500 rpm, or about 100 rpm to about 250 rpm, preferably about 24 rpm or above, which will increase the frequency range to obtain and utilize a desired amplitude and frequency. In some embodiments, the frequency of vibration may be about 0 Hz to about 200 Hz, about 50 Hz to about 150 Hz, or about 75 Hz to about 125 Hz. In general, it is anticipated that vibration frequencies greater than 160 Hz may tend to be less effective to minimize clot and/or biofilm formation, because at these and higher frequencies the degree of perceived vibrations will be diminished to the point that effective vibration will no longer be meaningfully discernible such that the introduction of mechanical energy to agitate the catheter in order to minimize clot-formation thereon will not be as effective. the motor can also include a speed controller to adjust or maintain a set frequency for delivering oscillatory vibration to the catheter. In yet other embodiments, the mechanical agitator includes a motor with an eccentric mass as an assembled unit. The mechanical agitator can also include more than one vibrating element (motor), positioned axial, obliquely or perpendicularly, or in combination of thereof relative to the long axis of the catheter. In these alternative embodiments, a similar frequency controller may be supplied.

In a preferred embodiment, the motor has a crank shaft (e.g. with an affixed eccentric mass) whose axis is oriented parallel to the long axis of the catheter at the point where it meets the hub connector 13. The orientation of the motor crankshaft with respect to the long axis of the catheter may be changed to alter the pattern of vibration applied to the catheter. In still other embodiments, there is a sealed interface around the motor and vibrating (eccentric) mass. The mechanical agitator 17 is configured to deliver mechanical motion, such as a vibration, fluctuation, oscillation or any combination thereof, from the hub connector 13, on or with which the mechanical agitator 17 is fixed or integrated, to the proximal end 15 of the catheter beginning where that catheter interfaces with the hub connector 13. The mechanical motion is then conducted along the length of the catheter 10 from the proximal end 15 to the distal end 16. The mechanical motion is sufficient to minimize the formation of obstructions within the one or more lumens 11, along the outer length of the catheter 10, and in or around the tip 12. Such mechanical motion also minimizes the formation and adherence of biofilm on the surface of the catheter within the circulatory system. In this manner, the risk of clot buildup and obstruction of the catheter is reduced.

In preferred embodiments, the mechanical motion supplied by the mechanical agitator 17 is sufficient to distribute motion along the entire length of the catheter 10 in order that an appreciable proportion of the vibratory energy introduced proximally by the mechanical agitator reaches the catheter tip 12. This ensures that the catheter will experience vibration along its entire length. Depending on the durometer and length of the catheter, the fluid carried therein (e.g. whether aqueous or gaseous-the latter as in the case of use for suctioning), and the surrounding environment (which may have damping effects on vibration), as well as the number and nature of any transitions in the surrounding environment (e.g. air-to-skin, skin-to-muscle, upon transitioning a vascular wall, etc.), the transmission of vibratory energy may be affected or diminished along the length of the catheter toward the tip 12, either linearly (if the catheter environment is substantially uniform along its length), or non-linearly (which may reflect discrete transitions). Catheter cross-section also may affect the transmission of vibratory energy; for example in the case of a variable cross-section along the length of the catheter. Alternatively, for a uniform-cross-section catheter having a modest (e.g. fewer than 3) significant environmental transitions and a modest length of, e.g. <40 or 50 cm, the transmission of vibratory energy from the vibratory energy may be substantially uniform along its length such that the vibration amplitude remains essentially constant, or diminishes by no more than, e.g. 10% or 5%, between the proximal end of the catheter and its tip 12.

The nature (i.e. degree of uniformity, versus degree of degradation) of vibratory-energy transmission along the length of the catheter based on the foregoing factors can be calibrated based on a variety of standard conditions that can be determined empirically in advance, as will be discussed in more detail below. In any event, because the accumulation of clot and biofilm at the outer surface of the catheter 10 is reduced when mechanical motion is applied along its full length, the propensity for clot growth and potentially infection is minimized. Also preferably, the delivery of mechanical motion from the mechanical agitator 17 is sufficient to reduce, and ideally prevent, the formation of obstructions not only at the catheter's 10 outer surface, but also within the inner lumen 11 along its full length to thereby maintain their patency.

DOES THIS PARAGRAPH MOVE? The positioning of the mechanical agitator 17 on the hub connector 13 upstream of the proximal end 15 of the catheter 10 generates a mechanical motion, such as vibration, fluctuation, oscillation or any combination thereof, that can be conducted along the entire length of the catheter 10 from the proximal end 15 to the distal end 16. The position of the mechanical agitator 17 on the hub connector 13 in this manner ensures that the mechanical motion is conducted from the proximal end 15 along the length of the catheter 10 to the distal end 16. Ideally, the mechanical agitator 17 Moreover, placement on or integration with the hub connector 13 positions the mechanical agitator 17 outside of the patient's body in-use.

In another embodiment (not shown), the mechanical agitator may be removably positioned on the catheter or on to an attachment mechanism capable of being removably positioned on the catheter. The attachment mechanism may include a clip, clamp, clasp, sleeve, male-female fastener, or any suitable combination thereof. The physical position of the mechanical agitator can be adjusted up and down the length of the catheter to ensure that mechanical motion is conducted appropriately along the length of the catheter in order that the desired degree of mechanical motion (such as vibration) is delivered to the tip 12 in-use. The longitudinal position of the mechanical agitator 17 relative to the catheter can be another of the standardized conditions against which calibration can be made empirically as described above. Alternatively, the longitudinal position can be adjusted based on feedback sensor data form a tip-embedded sensor, or based on tactile feedback and the experience of the clinician, both as also described above. For example, if the clinician observes that the vibration is unusually strong at the proximal end of the catheter, then the clinician can slide the attachment mechanism bearing the mechanical agitator down the catheter towards the distal end to redistribute the mechanical motion.

A controller coupled to the mechanical agitator 17 allows a user to adjust the amplitudes, frequencies and/or current of electrical energy delivered to the mechanical agitator 17 from its energy source to achieve a desired frequency and/or amplitude of vibration, in order to achieve a desired clinical condition. For example, portions of the catheter 10 external to the patient may vibrate at higher amplitudes than areas of the catheter 10 that are confined by or suspended in the vascular lumen, liquids, or larger anatomical entities (i.e., cardiac chambers, cavities, etc.). This is because portions of the catheter that are immersed in fluid or dense tissue will be more heavily damped than a free-floating segment of the catheter undamped through contact with external solid or liquid mass. In another example, the patient may be highly susceptible to thrombosis or have coagulopathies (i.e., altered blood coagulation) that require higher frequencies and/or lower amplitude to prevent clot formation than would ordinarily be required for a normal patient. Thus, in these and other instances, adjusting the amplitudes, frequencies and/or current of the electrical energy provided to the mechanical agitator 17 can change the force (i.e. amplitude) and/or frequency of vibrations or other mechanical motion delivered to the catheter. Further, the catheter 10 may be selected according to its dimensions (i.e., its length, diameter, and material density) such that adjusting the amplitudes, frequencies and/or current of the electrical energy provided to the mechanical agitator 17 may result in different vibratory affects at different parts of the catheter at the same time.

For example, for a given mechanical agitator 17 configured to deliver vibrations of a particular amplitude at the motor the effective amplitude of vibrations conducted through the catheter can be impacted by the density of the catheter material. A denser catheter material will tend to increase internal damping, thus reducing the effective vibratory amplitude experienced by the catheter when acted upon by a given mechanical agitator 17. On the other hand, a less dense catheter material will tend to decrease internal damping, thus increasing the effective vibratory amplitude experienced by the catheter based on the same mechanical agitator 17 with all other conditions being equal. It is contemplated that a system 100 as disclosed herein may be equipped with one of a selection of available catheters having a range of catheter-material densities, in order that the user may select an appropriate catheter density to achieve a desired vibratory amplitude for a given mechanical agitator 17 under operative conditions. Whereas vibratory amplitude can be adjusted as a function of catheter-material density based on other known or predicted damping factors (i.e. the environment in which the catheter is to be placed), vibration frequency can be readily adjusted simply by adjusting the frequency of vibrations. In the case of a micromotor supplying those vibrations, adjusting the voltage supplied to the motor can adjust its rotational velocity; i.e. its RPMs, And thus the frequency of delivered vibrations. By selecting an appropriate catheter-material density and adjusting to a desired RPM, one can tune both the amplitude-and frequency characteristics of desired vibrations. Alternatively or in addition to adjusting vibration amplitude via selection of an appropriate catheter-material density, one also may affect amplitude by adjusting the force motor actuations that produce vibrations. In the case of a motor with eccentric weight, for example, amplitude may be adjusted as a function of motor torque.

In one embodiment, a user may select pre-determined amplitudes, frequencies, current of the electrical energy provided from the power source, and/or orientation of the mechanical agitator 17 that are configured to provide a desired amount and characteristics of mechanical motion. The controller may also include an algorithm that prompts the user to enter specifications of the catheter 10, such as catheter length, number of lumens 11 and diameters thereof, and the catheter material, and uses said values to calculate ideal amplitudes, frequencies, current of the electrical energy from the power source, and/or orientation of the mechanical agitator 17 to provide the desired amount and characteristics of mechanical motion. In another embodiment, the user may manually adjust these parameters to impart a desired amount of mechanical motion to the catheter. To this end, the aforementioned parameters may be adjusted to continuously vibrate, oscillate, or fluctuate the catheter 10. Alternatively, they may be periodically adjusted to vibrate, oscillate or fluctuate the catheter 10 at predetermined time intervals, for example through the use of an algorithm programmed to the electrical energy source.

Such an algorithm also can be preprogrammed and stored in a memory embedded within or operationally coupled to the mechanical agitator 17, as a single algorithm or as one of a plurality of available and user-selectable algorithms, configured to operate the mechanical agitator 17 according to a predetermined motion-versus-time profile. Such an algorithm, for example, may apply variable and preprogrammed vibrations whose amplitude and frequency vary as a function of time. For example, the algorithm can be programmed to vibrate the catheter 10 continuously at a pre-determined amplitude, frequency and current for a predetermined interval of time (e.g., 2, 4, or 8 hours) after insertion, and then gradually adjust the amplitude and/or frequency of vibration after subsequent predetermined time intervals until the expiration of a predetermined final time interval, or until a clinician deactivates the mechanical agitator 17 to remove the catheter 10. The algorithm may be further configured to alert a user or a clinician after the expiration of the final time interval so that the caregiver can re-start the mechanical agitator 17 or remove the catheter 10.

Alternatively, such an algorithm can be programmed to vary those or other operational parameters as a function of one or more measured parameters, e.g. based on motion sensor data indicating whether the patient is ambulatory or immobile or the relative orientation of the patient (i.e., standing or laying down), sensor data relating to line pressure, blood pressure, pulse rate, blood counts, etc. For example, after a predetermined time interval, the controller can receive data regarding one or more of these parameters from sensors, such as the motion sensor 19 discussed above. The controller can compare the data to a predetermined threshold range for the parameter. The controller can then adjust at least one of the frequency or the amplitude of vibration, or the current of electrical energy provided to the mechanical agitator 17.

In further embodiments, such an algorithm may be programmed so that it will supply optimized motion-actuation performance to the catheter 10 based on a particular clinical condition, clinical indication, medical preconditions, blood properties, etc., wherein the algorithm can be selected and executed by the user having awareness of said clinical condition. Because a close inverse relationship exists between changes in viscosity and blood flow, the algorithm may also be pre-programmed to operate according to particular clinical scenarios or for particular fluids. Thus, for example, the algorithm may prompt a user to select a particular scenario, such as drug infusion, IV infusion line flushing, or ‘heavy’ fluid infusion (a term encompassing infusion of high molecular weight, high viscosity, and/or high thrombogenicity fluids like blood or contrast solutions), and then distribute mechanical motion along the length of the catheter 10 according to predetermined amplitude, frequency and energy parameters for the selected scenario. Alternatively, the algorithm may prompt a user to select a preprogrammed fluid type or to input the viscosity of a fluid to be administered, and then deliver mechanical motion along the length of the catheter 10 according to predetermined amplitude, frequency and energy parameters for that fluid or viscosity. The algorithm may also be programmed to continuously monitor infusion status through the catheter 10, such as through sensor data from a line pressure sensor or a fluid volume sensor configured to detect these parameters within the catheter, and stop current of energy to the mechanical agitator 17 when the line pressure or volume reaches a respective predetermined threshold value.

When executing an algorithm as discussed above, or any alternative algorithm, the transmission of vibratory energy along the length of the catheter based on that introduced by the mechanical agitator 17 can be calibrated based on a variety of standard conditions that can be determined empirically in advance. Such correlations can include empirical data reflecting a degree of vibratory energy (frequency and/or amplitude) at the tip 12 of the catheter that would be optimal to minimize the formation of clot and/or biofilm along the catheter length based on those conditions. Such standard conditions can include: catheter characteristics such as length, cross-section and durometer; use characteristics such as use as a PICC line, as a CVC line or some other known or standardized use; the predicted environmental transitions specific to the particular known or standardized use; the nature of the fluid (primarily its density) carried by the catheter in-use; and patient characteristics such as age, body-mass index, anticipated duration of implantation, implantation location (right-side heart, vena cava, etc.) and other clinical factors. All of these factors can be tabulated and correlated to yield a calibration table comprising calibration data that can be used to select the amplitude and frequency of vibratory energy to be introduced by the mechanical agitator 17 under the prevailing conditions, so that the amplitude and frequency of the vibratory energy exhibited at the tip 12 of the catheter will be appropriate to minimize or prevent the initial deposit or accumulation of biofilm or clot material along the entire length of the outer surface length of the catheter 10.

Because such accumulation on the outer surface of the catheter 10 is reduced when mechanical motion is applied, the propensity for clot growth is minimized. Also preferably, the delivery of mechanical motion from the mechanical agitator 17 is sufficient to reduce, and ideally prevent, the formation of obstructions not only at the catheter's 10 outer surface, but also within the inner lumen 11 to thereby maintain their patency. Both factors can be taken into account when calibrating a particular catheter under ranges of standardized conditions within the categories noted above (or other categories). In order to develop such calibration tables, empirical data may be collected from individual catheters under ranges of those conditions in simulated use environments wherein the degree of vibration transmission along their length, and the presence or absence of biofilm/clot-material formation is gauged. A limited set of such empirical determinations is disclosed in Example 1 herein.

In addition or as an alternative to empirical calibration based on standardized conditions as described above, the desired degree of mechanical motion (e.g. vibration) delivered to the tip 12 can be determined via a sensor 19 (such as motion sensor, e.g. an accelerometer) embedded in or adjacent to the tip. Specifically, a sensor 19 can be disposed in or adjacent to the tip 12 of the catheter in order to detect the vibrational state of the stip 12 in-use. The sensor may be connected via a wire that runs the length of (and can be embedded within or disposed in a lumen of) the catheter, for example, in order to deliver sensor signals to a controller for the mechanical agitator 17 (discussed below). When equipped with such a sensor 19, the vibrations introduced by the mechanical agitator 17 can be controlled or adjusted, either automatically or manually, to achieve a desired degree of mechanical motion at the tip 12 based on sensor feedback. In the case of manual adjustment, the degree of vibration can be reported on an output device such as a screen associated with the controller, so that the clinician can adjust the degree of vibration introduced by the mechanical agitator to achieve the desired sensor-measured vibration at the tip 12. And in the case of automatic adjustment, the aforementioned controller can execute a predetermined algorithm targeting the desired degree of sensor-measured vibration.

In a further alternative, the desired degree of mechanical motion delivered to the tip 12 can be determined via tactile feedback based on the experience and judgment of the clinician. For example, clinicians who routinely place PICC and CVC lines, for example, can become accustomed to the degree of tactile feedback in a vibrating catheter that in their experience corresponds to a desired degree of vibration or other mechanical motion delivered at the tip 12. The degree of familiarity with such procedures to make such judgments may benefit from sensor-calibration initially (along the lines described above), but over time may enable clinicians to make suitable judgments of tip-exhibited mechanical motion for a particular type of line that is placed under essentially the same conditions (e.g. a standard-size PICC line, placed in an average-sized mail or female patient, to deliver standard fluid), based on tactile feedback.

Still a further method to measure the degree of vibration to provide confirmation of or adjustment feedback for the delivery of suitable vibration energy can utilize external ultrasound, which will be able to detect the nature of vibrations along the catheter implanted within the patient, including at its tip 12.

In the embodiments shown in FIGS. 2 and 4 the mechanical agitator 17 is affixed to an outer surface of the hub connector 13 through a fixing element (not shown). The fixing element may include an adhesive, plastic welding, suction, a clasp, a sleeve, male-female fasteners, snap-in fasteners, other types of fasteners, or any combination thereof. The fixing element may also be integral with hub connector 13 and/or the mechanical agitator 17. In another embodiment, the fixing element may be configured to removably attach the mechanical agitator 17 to the hub connector 13. The fixing element should be sufficient to withstand the force of the applied mechanical motion to maintain the position of the mechanical agitator 17 on the hub connector 13. Alternatively, as noted above the mechanical agitator 17 can be integrated together and as part of the hub connector 13.

FIG. 5 is a schematic illustration of an embodiment of a system 100 for reducing formation of blood clots or biofilm on inner and outer surfaces of a tube for delivering fluid to a patient. The system 100 includes the catheter 10 inserted into a subclavian vein in a patient's body. Fluid connectors 14 are attached to hub connector 13. As depicted, when the mechanical agitator 17 is operative, it delivers mechanical motion from the hub connector 13 along the entire length of the catheter 10 from the proximal end 15 to the distal end 16 at the tip 12, wherein vibration eddies are conducted from the hub connector 13 through the solid-form material of the catheter 10, delivering those eddies all the way to the tip 12. The effect of conduction of mechanical motion along the entire length of the catheter 10 minimizes the accumulation and clot-nucleation of blood at both the outer and inner surfaces of the catheter 10, thus reducing (ideally eliminating) the formation of clots that may occlude either the vein in which the catheter 10 is placed or its lumen(s) 11. Such vibrations also will diminish the formation of biofilm on the surface of the catheter 10, thus minimizing the potential for biofilm-based infection.

### Example 1—Ex Vivo Study

An ex vivo test assessed the clotting inhibition of standard CVC lines, compared to the case when such lines were equipped with a mechanical agitator as herein disclosed. The experimental set up consisted of standard 8 Fr CVC lines divided into two groups: a control group comprised of four lines where no vibrational force was applied to the catheter, and an experimental group comprised of the remaining four lines where a vibrational force was applied to the catheter through a mechanical agitator affixed to an outer surface of the catheter's hub connector and operating at a frequency of 114 Hz+/−7 Hz. Accelerometers for motion-data collection were attached along the outer surface of each catheter in both groups; one adjacent to the proximal end of the catheter where vibration was introduced, and a second adjacent to the tip of the catheter in order to measure the degree of oscillatory-energy transmission along the catheter length to its tip. All catheters of the control-and experimental groups were then placed concentrically through respective rubber sleeves to simulate vascular placement of the CVC line, and then exposed to and/or submerged in a test medium of bovine blood, which filled the annular space between the outer surface of each catheter and its surrounding rubber sleeve. The control-and experimental group catheters were then filled with bovine blood. Conditions favoring thrombus formation inside and outside the control-and experimental group catheters were simulated by use of human thrombin added to the bovine blood in test medium and in the catheters. Human thrombin was added to the bovine blood serving as the test medium surrounding the outside of each catheter, and then within in the catheters in the experimental group after the respective mechanical agitator was activated.

The CVC lines for both the control-and experimental groups were placed on-test for 30 minutes to 1 hour, after which the respective catheters were examined for clotting.

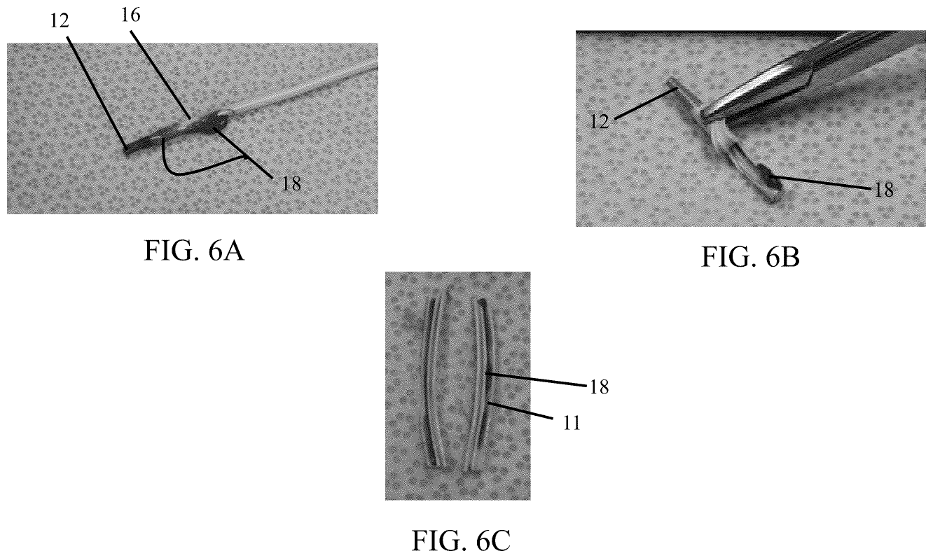

Clotting 18 was observed along the outer surface of the tip 12 and the distal end 16 of the catheters in the control group following the test, as seen for one of the control-group catheters pictured in FIG. 6A. As shown in FIGS. 6B and 6C, which are representative of the control-group catheters, clotting 18 also occurred within sections of the lumen of the control group.

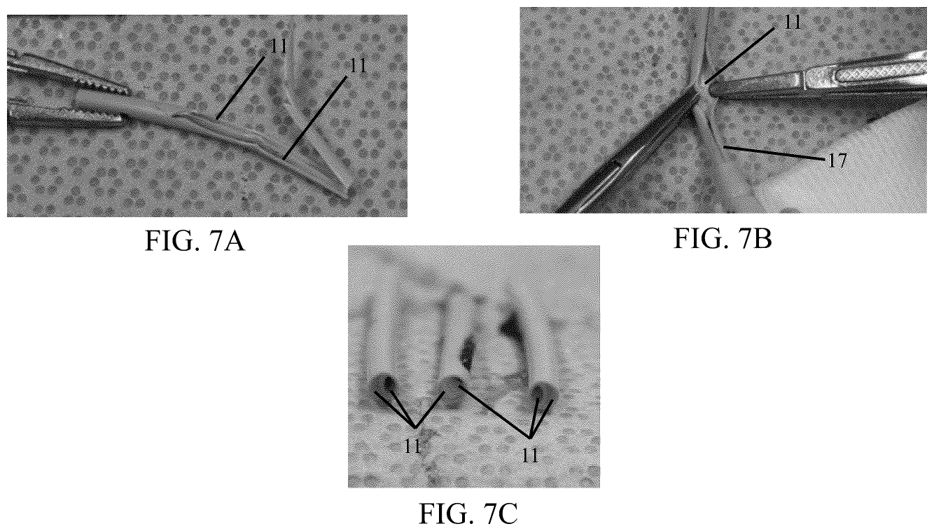

In contrast, the catheters of the experimental group exhibited no clinically significant clotting either along sections of the catheter lumen or at their outer surfaces. FIGS. 7A-7C are representative of the catheters in the experimental group following the test.

Accordingly, the distribution of vibrational force from a mechanical agitator affixed to the hub connector of a catheter effectively inhibited the formation of clots within the catheter lumen.

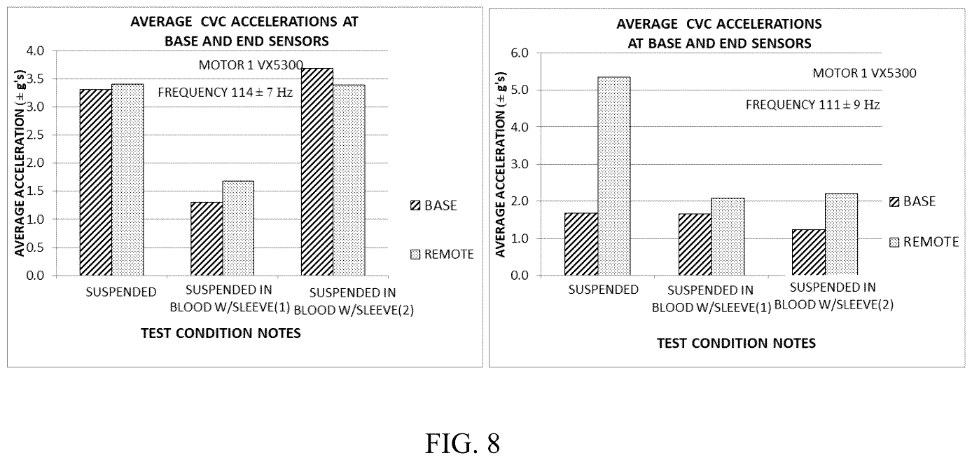

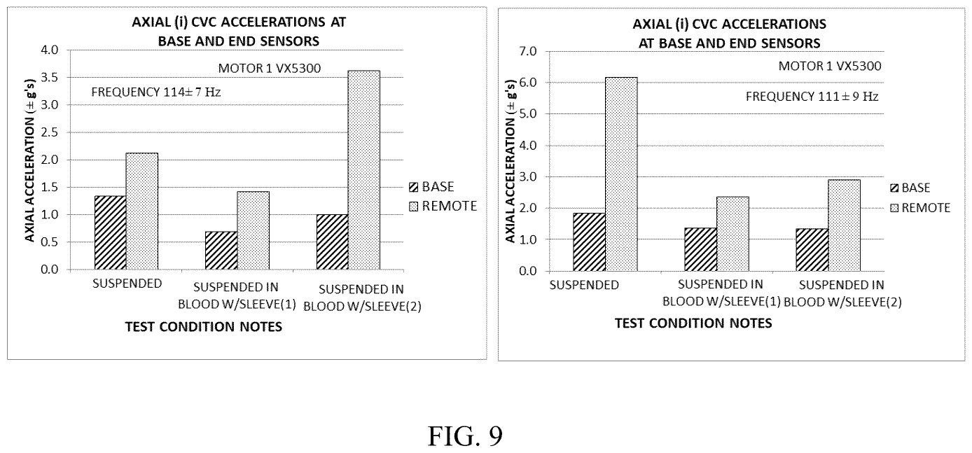

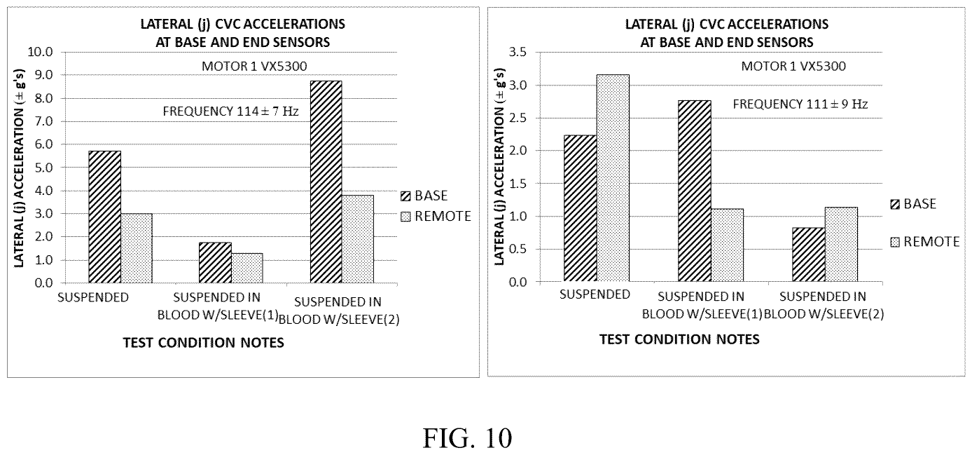

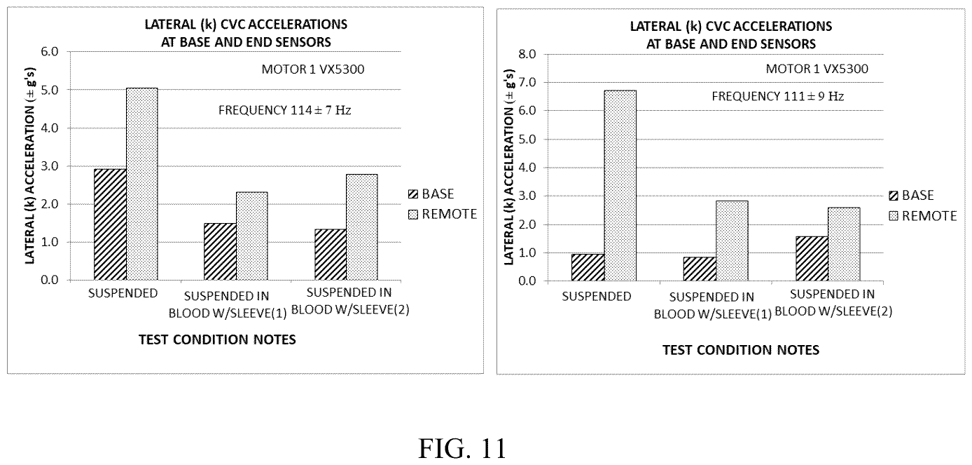

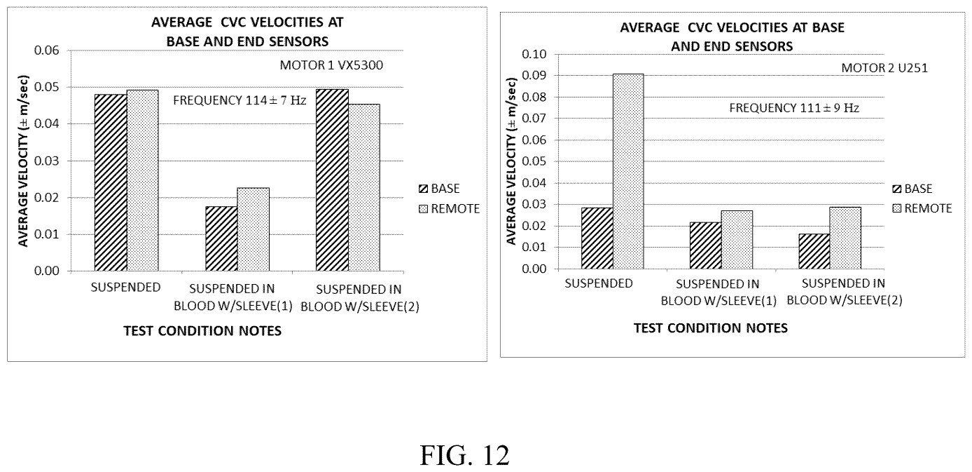

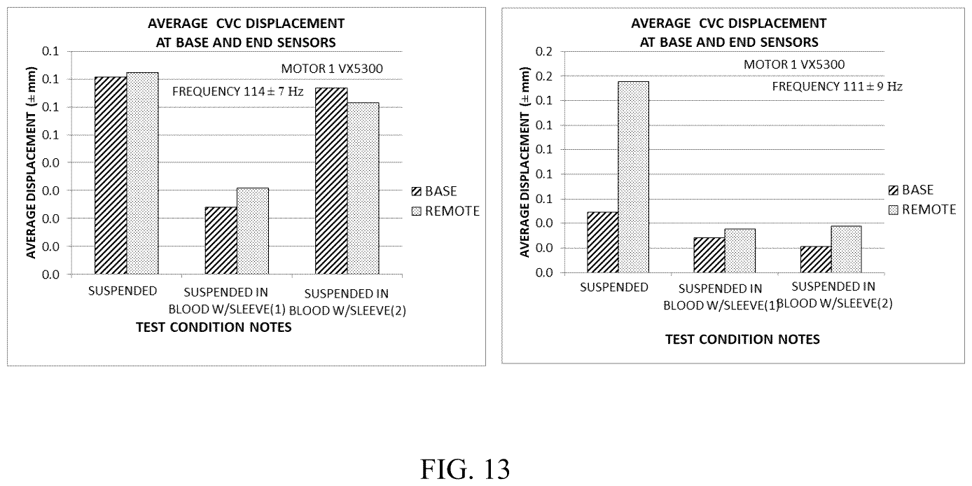

FIGS. 8-13 illustrate summarize accelerometer data obtained during the aforementioned experiments in relation to the catheters on-test at two different frequency setpoints: 114+/−7 Hz, and separately at 111+/−9 Hz. As seen in FIG. 8, correlations of average acceleration observed at the tip of each catheter (“REMOTE”—right-hand bar in each graph) compared to at the proximal end of that catheter (“BASE”—left-hand bar in each graph) were established based on the prevailing conditions during the test for the three test conditions: Suspended in Air; Suspended in Blood w/Sleeve(1); Suspended in Blood w/Sleeve(2). Sleeve(1) was a first polymer material sleeve, whereas Sleeve(2) was a second polymer material sleeve having different physical properties. As seen in the figure, under the prevailing conditions of each test a correlation can be established between the average acceleration introduced at the proximal end of the catheter (“BASE”) and that observed at the tip of that catheter (“REMOTE”) in all cases. The relative correlations change depending on the surrounding environment, but in each case it is possible to establish a correlation between the REMOTE portion of the catheter near the tip, and the BASE portion near the proximal end where vibratory energy will be supplied. Similar correlations can be determined empirically as described above across ranges of standardized conditions to construct one or more calibration table(s) as also described above, to correlate input energy (frequency/magnitude) at the mechanical actuator to observed energy (frequency/amplitude) at or adjacent the tip. FIGS. 9-11 depict, respectively, the specific orthogonal-component accelerations observed both at “BASE” and “REMOTE” during each test (longitudinal axis i, plus two lateral axes j and k). FIGS. 12 and 13 reflect additional measurements of average velocity and average displacement, respectively, obtained at each of the “BASE” and “REMOTE” locations. These data also can be correlated to a degree of mechanical energy delivered at the tip (“REMOTE”) that is observed empirically to be effective to minimize the formation of cloth and/or biofilm along the catheter in the construction of a calibration table as herein described.

From the above description, those in the art will perceive improvements, changes and modifications. Such improvements, changes and modifications are within the skill of one in the art and are intended to be covered by the appended claims.

1. A system for minimizing formation of clot in a medical tube, the system comprising:
a hub connector configured to provide fluid communication between a medical tube when attached thereto and one or more fluid sources; and
a mechanical agitator adapted to be disposed at an outer surface of the hub connector and configured to deliver oscillatory mechanical energy thereto in order that said oscillatory mechanical energy further will be delivered along a full length of the medical tube, to a tip thereof.

2. The system of claim 1, wherein the medical tube is a catheter.

3. The system of claim 2, wherein the catheter is a CVC line or a PICC line.

4. The system of claim 1, wherein the mechanical agitator is removably affixed to the hub connector.

5. The system of claim 1, wherein the mechanical agitator is integrated with the hub connector.

6. The system of claim 5, wherein the hub connector is integrated with the medical tube and the fluid source connector to form an integrated system.

7. The system of claim 1, wherein the medical tube comprises a first lumen and a second lumen.

8. The system of claim 7, wherein the system comprises a first fluid source and a second fluid source, wherein a first fluid from the first fluid source flows through the hub connector into the first lumen, and wherein a second fluid from the second fluid source flows through the hub connector into the second lumen.

9. The system of claim 1, wherein the mechanical agitator comprises a motor having an eccentric mass connected to a crank shaft thereof.

10. The system of claim 9, wherein the hub connector conducts vibratory mechanical energy supplied from said motor to said medical tube substantially uniformly along a length of the medical tube.

11. A method of minimizing formation of clot on inner and outer surfaces of a medical tube, the method comprising:
attaching a proximal end of the medical tube to a hub connector having a mechanical agitator integrated with the hub connector and configured to deliver oscillatory mechanical energy;
inserting a distal end of the medical tube into a blood vessel of a patient; and
activating the mechanical agitator to deliver oscillatory mechanical energy to the medical tube, via said hub connector, so that said oscillatory mechanical energy is delivered along an entire length of the medical tube to a tip thereof implanted within the blood vessel.

12. The method of claim 11, further comprising attaching at least one fluid source to the hub connector.

13. The method of claim 11, wherein a controller is coupled to the mechanical agitator and configured to control an amplitude of the oscillatory mechanical energy, and/or a frequency of the oscillatory mechanical energy.

14. The method of claim 13, wherein the controller executes an algorithm to carry out the following steps:
(a) after activating the mechanical agitator, operate the mechanical agitator continuously at a predetermined frequency for a time interval; and
(b) after the time interval, adjust at least one of the frequency, or the amplitude.

15. The method of claim 14, wherein steps (a) and (b) are repeated until the expiration of a predetermined final time interval or until a user deactivates the mechanical agitator.

16. The method of claim 13, wherein the controller executes an algorithm to carry out the following steps:
(a) after activating the mechanical agitator, operate the mechanical agitator continuously at a predetermined frequency for a time interval; and
(b) after the time interval, receive data regarding at least one parameter from one or more sensors, and compare that data to a predetermined threshold range for the at least one parameter;
(c) adjust at least one of the frequency, or the amplitude; and
(d) repeat steps (a)-(c) until the expiration of a predetermined final time interval.

17. The method of claim 16, wherein the at least one parameter is selected from the group consisting of motion-sensor data, line pressure, blood pressure, pulse rate, blood counts, fluid volume, or a combination thereof.

18. The method of claim 13, wherein the controller executes an algorithm to adjust the mechanical agitator according to predetermined calibration data correlating the amplitude and/or vibration of oscillatory mechanical energy delivered by the mechanical agitator to that observed at a tip of the medical tube across a range of one or more standardized conditions.

19. The method of claim 18, wherein said algorithm selects values among the range(s) of the one or more standardized conditions that correspond(s) to prevailing conditions in-use, and adjusts the mechanical actuator in order that a predetermined desired degree of oscillatory mechanical energy is achieved at the tip based on the prevailing conditions.
